(19) United States
(12) Patent Application Publication
(10) Pub. No.: US 2014/0031275 A1
(43) Pub. Date: Jan. 30, 2014

(54) METHODS OF ERADICATING BACTERIAL CELL POPULATIONS

(71) Applicants: Kim LEWIS, Newton, MA (US); Brian CONLON, Boston, MA (US); Mark L. NELSON, Norfolk, MA (US); Michael P. POLLASTRI, Waltham, MA (US); Thomas A. DAHL, Madison, CT (US)

(72) Inventors: Kim LEWIS, Newton, MA (US); Brian CONLON, Boston, MA (US); Mark L. NELSON, Norfolk, MA (US); Michael P. POLLASTRI, Waltham, MA (US); Thomas A. DAHL, Madison, CT (US)

(73) Assignee: NORTHEASTERN UNIVERSITY, Boston, MA (US)

(21) Appl. No.: 14/043,362

(22) Filed: Oct. 1, 2013

Related U.S. Application Data
(63) Continuation of application No. PCT/US12/31882, filed on Apr. 2, 2012.
(60) Provisional application No. 61/470,864, filed on Apr. 1, 2011.

(51) Int. Cl. A61K 31/437; A61K 31/5377; A61K 31/343

(52) U.S. Cl. CPC A61K 31/437; A61K 31/343; A61K 31/5377
USPC 514/2.8; 514/287; 514/236.8

(57) ABSTRACT

Disclosed herein are methods and compositions for the eradication of bacterial infections. In particular, methods and compositions are disclosed for the eradication of persister and slow growing bacterial cell populations. In particular embodiments, the methods and compositions disclosed herein are useful for eradication of biofilms.

### RELATED APPLICATIONS

This application is a Continuation of International Application No. US12/31882, filed on Apr. 2, 2012, entitled “Methods of Eradicating Bacterial Cell Populations”, which claims the benefit of U.S. Provisional Patent Application No. 61/470,864, filed on Apr. 1, 2011, entitled “Methods of Eradicating Bacterial Cell Populations”, each of these applications is incorporated by reference herein in its entirety.

### STATEMENT CONCERNING GOVERNMENT RIGHTS IN FEDERALLY-SPONSORED RESEARCH

This invention was made with United States government support under Grant No. T-RO1AI085585 awarded by the National Institutes of Health. The United States government has certain rights in this invention.

### INCORPORATION BY REFERENCE

All patents, patent applications and publications cited herein are hereby incorporated by reference in their entirety in order to more fully describe the state of the art as known to those skilled therein as of the date of the invention described herein.

### FIELD OF THE INVENTION

The present invention relates to the field of medicine. More specifically, the present invention relates to treatment of infections and eradication of drug-tolerant infections.

### BACKGROUND OF THE INVENTION

Recalcitrant chronic infections affect a significant portion of the population. Whenever the efficiency of the immune system is decreased, an infection can become chronic. Examples of such cases are immunocompromised patients, or patients having an infection that forms a biofilm limiting the penetration of immune components—in abscesses, infections of heart valves, osteomyelitis, or on indwelling medical devices. In some cases, infection is caused by drug-resistant bacteria that grow on these devices, as well as in and around tissue in contact with indwelling devices. However in most cases, recalcitrance of a chronic infection is not caused by drug resistance. Rather, slow-growing bacterial populations produce drug-tolerant persister cells that are difficult to eradicate with existing antibiotics (Lewis, K., (2010) Persister cells. Annu Rev Microbiol 64: 357-372). When antibiotic concentrations drop, persister cells regrow and repopulate the biofilm.

Non-growing stationary bacterial populations and slow-growing biofilms are difficult to kill. Moreover, stationary populations of gram-positive pathogens are especially tolerant to antibiotics. Rifampicin, an inhibitor of RNA polymerase, is known to be the most effective bactericidal antibiotic acting against M. tuberculosis. However, killing in vitro in this manner requires several days of incubation. Rifampicin has not been known to kill cells in stationary populations of other gram-positive pathogens in vitro.

Thus, there remains a need for therapies capable of killing persister cells. In particular, there remains a need for therapies that eliminate bacteria of all types, including rapidly growing cells (e.g., exponentially growing cells), cells in stationary growth phase, and persister cells.

### SUMMARY OF THE INVENTION

The present methods and compositions disclosed herein are useful for treating bacterial infections and eradicating infections of indwelling devices such as catheters, heart valves, and other such devices. Such devices are associated with an increased risk of infection. Acyldepsipeptides (“ADEP”) in combination with one or more antibiotics can be used in accordance with the present disclosure to eradication bacterial cultures in a matter of days. Eradication can be achieved by a combination of ADEP with antibiotics such as rifampicin or oxacillin. In addition, the disclosed methods and compositions decrease the duration of treatment for gram-positive diseases, such as those caused by Staphylococcus aureus.

There are at least two unsolved problems in the field of antimicrobials: (1) developing effective approaches for combating drug-resistant pathogens and (2) treating chronic infections that are antimicrobial-tolerant. Regarding drug resistance, many antibiotics have been developed over the years that are effective against most disseminating infections. New compounds for combating drug resistance are also forthcoming.

However, currently there is no therapeutic capable of eradicating chronic infections. Currently available antibiotics are effective because the actions of the antibiotic and that of the immune system are complementary. Antimicrobials generally eliminate the majority of a pathogenic population or stop the growth of cells that make up a bacterial population, and the immune system kills the population that remains.

Furthermore, when biofilms are present, the exopolymer matrix of the biofilm protects the pathogen by preventing components of the immune system from accessing the pathogen. This results in chronic infection, which is difficult to treat. Particularly difficult to treat chronic infections include, for example, endocarditis, osteomyelitis, cystic fibrosis, abscesses, infections of indwelling devices, and dental diseases. As used herein, the term “indwelling device” means an instrument that is invasive and placed either permanently or temporarily into the body. One reason that such infections are difficult to treat is that antibiotics require active targets to be effective. However, targets in dormant cells, such as those in biofilms, are mainly inactive, rendering antibiotics alone ineffective against these populations (see, e.g., Keren, I., D. Shah, A. Spoering, N. Kaldalu & K. Lewis, (2004b) J Bacteriol 186: 8172-8180). Once antibiotic concentrations fall below a certain threshold, persister cells repopulate the biofilm, causing a relapsing chronic infection.

Accordingly, aspects disclosed herein relate to methods of killing cultures or populations of persister cells and stationary phase cells, as well as exponentially growing bacterial cells. In certain embodiments, the bacterial cells are gram-positive bacteria. In other embodiments, the bacterial cells are in a stationary growth phase. In still other embodiments, the bacterial population is a mixture of exponentially growing cells, cells in stationary phase, and persister cells. Certain methods disclosed herein comprise administering an effective amount of ADEP in combination with an effective amount of an antibiotic. In certain embodiments, the antibiotic is rifampicin. In other embodiments, the antibiotic is oxacillin.

In certain embodiments disclosed herein, the ADEP and the antibiotic are administered to the indwelling device in an effective amount of about 0.5 mg to about 5,000 mg per day. In other embodiments, the indwelling device is impregnated with ADEP alone in an effective amount of about 0.5 mg to about 5,000 mg.

Additional aspects include methods of treating chronic or relapsing infections by administering an effective amount of ADEP in combination with an effective amount of at least one antibiotic. In certain embodiments, the ADEP and antibiotic are provided in compositions comprising about 0.5 mg to about 5,000 mgmgmg.

In one or more embodiments, a method of treating a bacterial infection is disclosed herein. The method comprises administering to a subject an effective amount of a compound having the structure:

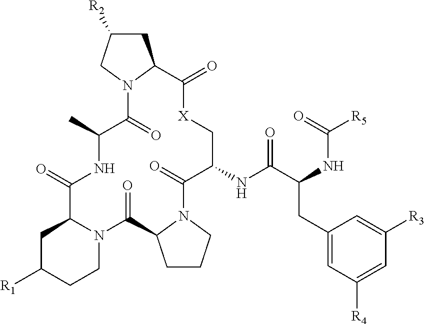

wherein R1, R2, R3, and R4 are each independently H, alkyl, aryl, or halogen, R5 is hydrogen, alkyl, alkenyl, or aralkyl where H may be hydrogen, deuterium, or tritium, and wherein X is oxygen or NH, or a pharmaceutically acceptable salt thereof. Moreover, an effective amount of one or more antibiotics is administered in combination with Formula I, wherein the combination of Formula I and one or more antibiotics kills bacterial cells.

In one or more aspects, the subject is treated for at least 2 days with the combination.

In an aspect of any of the embodiments, the effective amount of the compound is selected from the range of 0.5 mg to 250 mg. In still other aspects, the effective amount of the one or more antibiotics is selected from the range of 0.5 mg to 250 mg.

In an aspect of any of the embodiments, the one or more antibiotics are selected from rifampicin, oxacillin, amphotericin, ampicillin, b-lactam antibiotics, rifamycin group antibiotics, ciprofloxacin, erythromycin, macrolides, methicillin, metronidazole, ofloxacin, penicillin, streptomycin, tetracycline, vancomycin, and combinations thereof.

In another aspect of any of the embodiments, the bacterial cells can be resistant to an acyldepsipeptide. The bacterial cells can be persister cells. In the alternative, the bacterial cells can be persister cells, cells in stationary growth phase, or rapidly growing cells. In one aspect, the bacterial cells are gram positive. In another aspect, the bacterial cells are gram-negative. In one or more aspects of any embodiment, the bacterial cells are selected from MRSA S. aureus, VRE E. faecalis, S. pneumoniae, S. epidermidis, and combinations thereof.

In one or more aspects of any embodiment, the composition further comprises polymyxin B nonapeptide. In another aspect, the composition further comprises MDR inhibitor.

Disclosed herein are methods of eradicating bacteria from a device. The device is contacted with a combination of a compound having the structure:

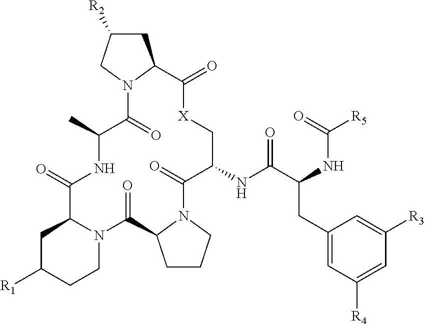

wherein R1, R2, R3, and R4 are each independently H, alkyl, aryl, or halogen, R5 is hydrogen, alkyl, alkenyl, or aralkyl where H may be hydrogen, deuterium, or tritium, wherein X is oxygen or NH, or a pharmaceutically acceptable salt thereof. The device is contacted with at least one antibiotic. The combination of the compound and at least one antibiotic is effective to kill the bacteria on the device.

In an aspect of the embodiment, the device is an implantable device.

In an aspect of any of the embodiments, the combination comprises an effective amount of the compound and an effective amount of at least one antibiotic to eradicate the device.

Disclosed herein is a formulation for killing persister bacterial cells. The formulation includes an effective amount of a compound having a structure:

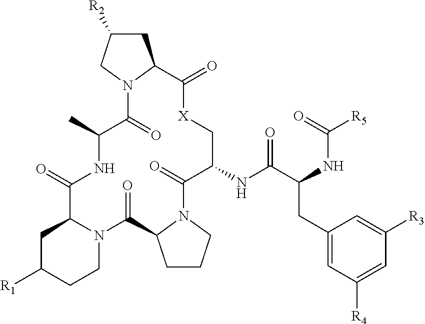

wherein R1, R2, R3, and R4 are each independently H, alkyl, aryl, or halogen, R5 is hydrogen, alkyl, alkenyl, or aralkyl where H may be hydrogen, deuterium, or tritium, wherein X is oxygen or NH, or a pharmaceutically acceptable salt thereof. The formulation further comprises an effective amount of at least one antibiotic.

In any of the embodiments, the at least one antibiotic is rifamycin. In one aspect, the compound is ADEP4.

In one or more aspects, the compound is L-proline, 3-fluoro-N-[(2E)-1-oxo-2-hepten-1-yl]-L-phenylalanyl-L-seryl-L-prolyl-(2S)-4-methyl-2-piperidinecarbonyl-L-alanyl-, (6→2)-lactone.

In certain aspects, ADEP and antibiotic are provided in formulations comprising about 0.5 mg to about 5,000 mg.

Additional aspects disclosed herein relate to the use of the compounds of Formula I to eradicate persister cell populations. In particular aspects, the methods comprise administering to a subject an effective amount of a compound having the structure:

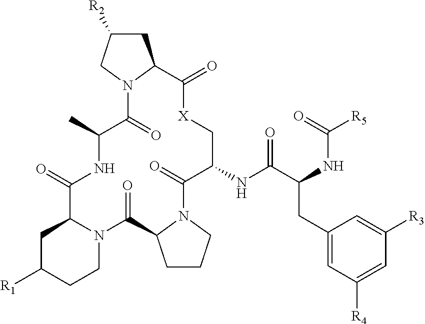

wherein R1, R2, R3, and R4 are each independently H, alkyl, aryl, or halogen,
R5 is hydrogen, alkyl, alkenyl, or aralkyl where H may be hydrogen, deuterium, or tritium, and wherein X is oxygen or NH, or a pharmaceutically acceptable salt thereof; wherein compound of Formula I kills bacterial cells.

In certain embodiments, the subject is treated for at least 2 days with the combination. In other embodiments, the effective amount of the compound is selected from the range of 0.5 mg to 250 mg. In still other embodiments, the bacterial cells are gram positive. In particular embodiments, the bacterial cells are selected from MRSA S. aureus, VRE E. faecalis, S. pneumoniae, S. epidermidis, and combinations thereof. In still further embodiments, the bacterial cells are gram-negative. In particular embodiments, the composition further comprises polymyxin B nonapeptide. In more particular embodiments, the composition further comprises MDR inhibitor.

### BRIEF DESCRIPTION OF THE FIGURES

FIG. 1 is a graph showing antibiotic action against stationary state S. aureus. SA113.

FIG. 2A is a bar graph that shows the sterilization of a stationary culture MSSA in the presence of ADEP 10c (1×MIC) and oxacillin (100×MIC), linezolid (10×MIC) or rifampicin (10×MIC).

FIG. 2B is a bar graph that shows wild-type SH1000 and a clpP-deletion mutant in the presence of various antibiotics at 10×MIC.

FIG. 2C Sterilization of a stationary MRSA 37 culture, in the presence of ADEP 10c (1×MIC) and linezolid (10×MIC) or rifampicin (10×MIC). Black line represents the limit of detection

FIG. 3 is a schematic showing the structure of ADEP 4 and ADEP 10.

FIG. 4A is a schematic showing the synthesis of ADEP 4, ADEP 10c, and hybrid analogs.

FIG. 4B is a schematic showing the synthesis of aza-analogs of ADEP 4, ADEP 10c, and hybrid analogs.

### DETAILED DESCRIPTION OF THE INVENTION

### 1. General

Disclosed herein are composition and methods for eliminating bacterial cell populations. In particular embodiments, the bacterial cell infections are associated with chronic or persistent infections relating to biofilms that comprise persister cells. In some instances, a subject is administered the compositions to treat the bacterial infection. For example, a patient could be administered a composition comprising ADEP and at least one antibiotic in amounts effective to eliminate the infection. In other embodiments, the disclosed compositions are applied to a material to eradicate the material of bacterial cells. For example, a device could be eradicated prior to use in surgical procedures. The compositions and methods disclosed herein can be used on bacterial cells in exponential growth phase and in stationary phase. In addition, the compositions and methods can also be used to treat persister bacterial cells.

ADEP, produced by an Actinomycete, was discovered twenty-six years ago (see, e.g., U.S. Pat. No. 4,492,650). Early researchers abandoned the compound after finding that it had good activity against gram-positive bacteria, but not against gram-negative bacteria. There are at least six ADEP molecules that are known to the art (see, e.g., Brotz-Oesterhelt et al. (2005) Nature Medicine 11: 1082-1087). The structures of these molecules are shown below.

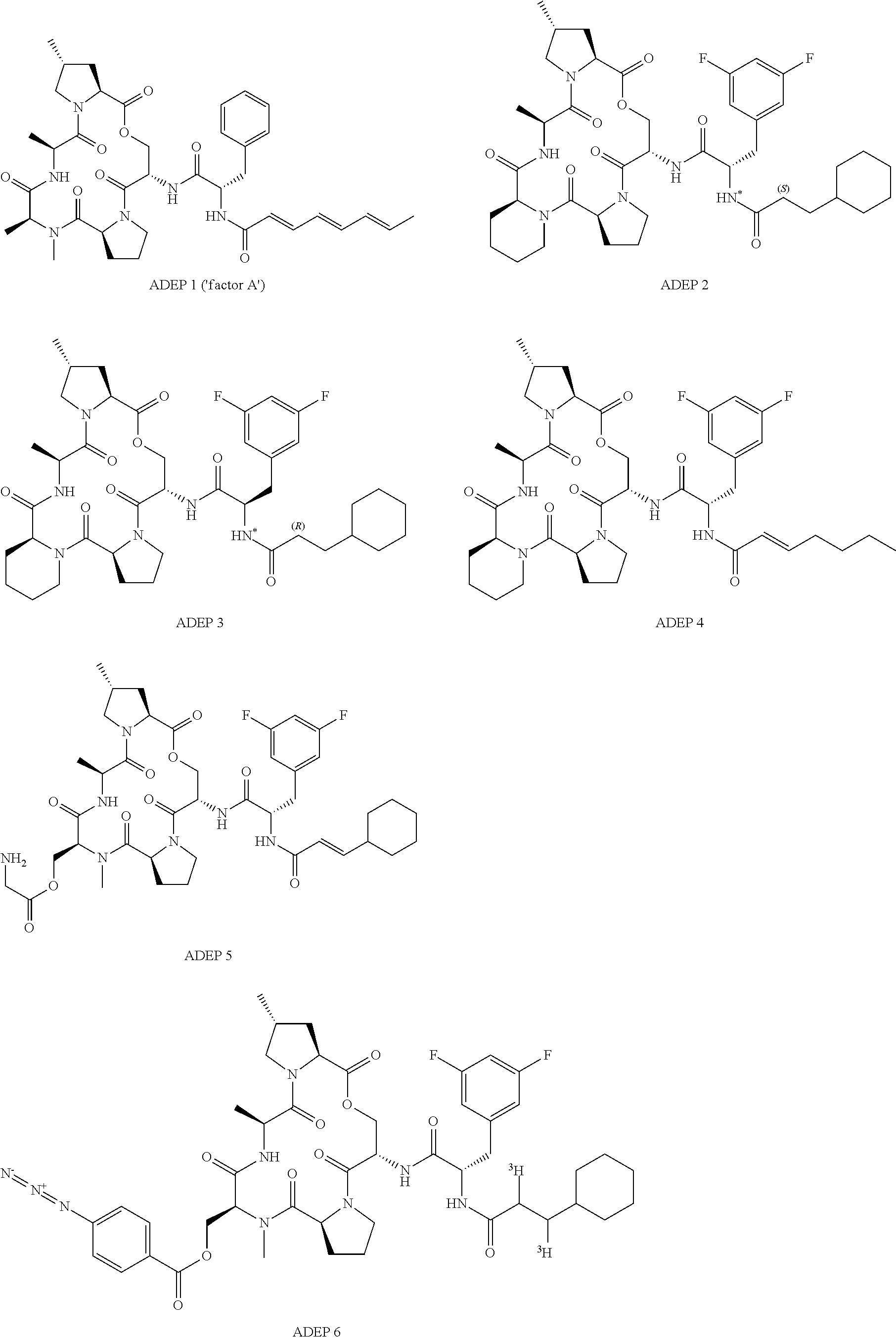

ADEP compounds activate the ClpP protease. The protease, in turn, degrades proteins necessary for bacterial cell survival, thereby killing bacterial cells that are sensitive to ADEP. In particular, ADEP4 was found to be safe and effective in several animal models of uncomplicated disseminating infection caused by S. aureus, and E. faecalis (see id.). However, it appeared that resistance to ADEP occurred at an alarmingly high rate due to null mutations in non-essential ClpP. Issues related to the high rate of resistance to ADEP once again resulted in the abandonment of ADEP research.

The disclosed compositions and methods utilize a heretofore unknown characteristic of ADEP—the ability of these compounds to eradicate persister and slow growing bacterial cells. In addition, the disclosed compositions and methods overcome issues relating to ADEP resistance. In particular, the disclosed compositions and methods allow for the use of ADEP with antibiotics to eradicate bacterial infections, including rapidly growing cells (e.g., exponentially growing cells), cells in stationary growth phase, and persister cells, without causing high levels of antimicrobial resistance. In certain embodiments, a combination of ADEP4 and 10c with one or more antibiotics has been found to be effective at eradicating bacterial infections. Furthermore, certain disclosed compositions and methods utilize ADEP to eradicate slow growing bacterial cells. In particular embodiments, combinations of ADEP compounds are used to eradicate bacterial cells.

In particular aspects, the compositions and methods disclosed herein relate to eradicating persister bacterial cells. Persister cells are dormant phenotypic variants of wild-type cells that are tolerant to antibiotics (Lewis, K., (2010) Persister cells. Annu Rev Microbiol 64: 357-372). All forms of pathogens form persisters, which make up 10⁻⁵ of a growing bacterial population (Lewis 2010). This number increases to 1% in stationary cultures of E. coli (Keren, I., N. Kaldalu, A. Spoering, Y. Wang & K. Lewis (2004a) FEMS Microbiol Lett 230: 13-18). It is speculated that in gram-positive S. aureus, a stationary culture is made of persister cells that are nearly insensitive to antibiotics. In addition, it appears likely that there are many independent, redundant mechanisms of persister formation, and that these specialized survivor cells lack targets that can be exploited for drug development (see, e.g., Hansen, S., K. Lewis & M. Vulic, (2008) Antimicrob Agents Chemother.; LaFleur, M. D., Q. Qi & K. Lewis, (2010) Antimicrob Agents Chemother 54: 39-44). In other words, persister cells represent a potential complicating factor in many infections and in biofilms.

As disclosed herein, persister cells can be killed using the compositions disclosed herein. An effective amount of ADEP can be used to corrupt cell functions without requiring energy input. In particular, two ADEP derivatives, ADEP 4 and 10c (“L-proline, 3-fluoro-N-[(2E)-1-oxo-2-hepten-1-yl]-L-phenylalanyl-L-seryl-L-prolyl-(2S)-4-methyl-2-piperidinecarbonyl-L-alanyl-, (6→2)-lactone”), can be used to eradicate stationary cultures of S. aureus. Furthermore, the compositions comprise an effective amount of one or more antibiotics to eradicate the growing phase bacterial cells. Although both antibiotics and ADEP are useful to kill bacterial cells, the disclosed compositions and methods successfully eradicate bacterial cell populations.

### 2. Compounds

Disclosed herein are ADEP compounds for use in methods of eradicating, treating, or killing bacterial cell populations. Compounds of the structure

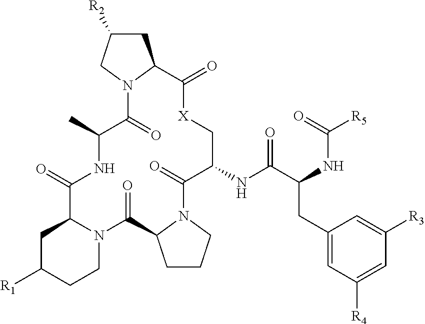

wherein R1, R2, R3, and R4, are each independently H, alkyl, aryl, halogen, and R5 is hydrogen, alkyl, alkenyl, or aralkyl where H may be hydrogen, deuterium, or tritium. In particular embodiments, R1 is alkyl. In other embodiments, R1 is CH₃. In certain embodiments, R5 is alkyl. In particular embodiments, R5 is an alkyl having one to six carbons. In other embodiments, R3 is fluorine. In other embodiments, R2 and R4 are hydrogen.

In certain embodiments, X is oxygen or NH. In certain embodiments, the compound is provided with a pharmaceutically acceptable salt thereof. The phrase “pharmaceutically acceptable salt,” as used herein, means those salts of compounds that are safe and effective for use in a subject. Pharmaceutically acceptable salts include salts of acidic or basic groups present in compounds. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts, reference is made to Berge et al., 66 (1977) J. Pharm. Sci. 1-19, incorporated herein by reference.

In particular aspects, one or more of the compounds are used to eradicate bacterial cells. In some embodiments, one or more compounds used in the compositions are ADEP1, ADEP2, ADEP3, ADEP4, ADEP5, ADEP6, and 10c, which may be used alone or in combination with antibiotics to eradicate bacterial cells. In particular embodiments, one ADEP compound is used in the composition. In certain embodiments, ADEP4 and 10c are useful to eradicate bacterial cell populations. In more particular embodiments, ADEP4 is used to eradicate bacterial cell populations.

In particular embodiments, X is O, R1 is methyl, R2 is hydrogen, and R5 is 1-hexene (e.g., hexylene, butyl ethylene), while R3 and R4 are fluorine. In other embodiments, X is O, R1 is hydrogen, R2 is methyl, and R5 is 1-hexene (e.g., hexylene, butyl ethylene), while R3 is fluorine and R4 is hydrogen. In certain embodiments, X is NH, R1 is methyl, R2 is hydrogen, R3 is fluorine, R4 is hydrogen, and R5 is 1-hexene (e.g., hexylene, butyl ethylene). In particular embodiments, X is NH, R1 is hydrogen, R2 is methyl, R3 is fluorine, R4 is fluorine, and R5 is 1-hexene (e.g., hexylene, butyl ethylene). In some embodiments, X is NH, R1 is methyl, R2 is hydrogen, R3 is fluorine, R4 is fluorine, and R5 is 1-hexene (e.g., hexylene, butyl ethylene). In other embodiments, X is NH, R1 is hydrogen, R2 is methyl, R3 is fluorine, R4 is hydrogen, and R5 is 1-hexene (e.g., hexylene, butyl ethylene).

In some aspects, the compositions disclosed herein do not include compounds wherein X is O, R1 is methyl, R2 is hydrogen, R5 is 1-hexene (e.g., hexylene, butyl ethylene), while R3 and R4 are fluorine. In other aspects, the compositions do not include compounds wherein X is O, R1 is hydrogen, R2 is methyl, and R5 is 1-hexene (e.g., hexylene, butyl ethylene), while R3 is fluorine and R4 is hydrogen. Aspects of compositions disclosed herein do not include compounds wherein X is NH, R1 is methyl, R2 is hydrogen, R3 is fluorine, R4 is hydrogen, and R5 is 1-hexene (e.g., hexylene, butyl ethylene). In some aspects, the disclosed compositions do not include compounds wherein X is NH, R1 is hydrogen, R2 is methyl, R3 is fluorine, R4 is fluorine, and R5 is 1-hexene (e.g., hexylene, butyl ethylene). Aspects of compositions disclosed herein do not include compounds wherein X is NH, R1 is methyl, R2 is hydrogen, R3 is fluorine, R4 is fluorine, and R5 is 1-hexene (e.g., hexylene, butyl ethylene). In some aspects, the compositions disclosed herein do not include compounds wherein X is NH, R1 is hydrogen, R2 is methyl, R3 is fluorine, R4 is hydrogen, and R5 is 1-hexene (e.g., hexylene, butyl ethylene).

In other embodiments, the compositions can comprise compounds of the structure

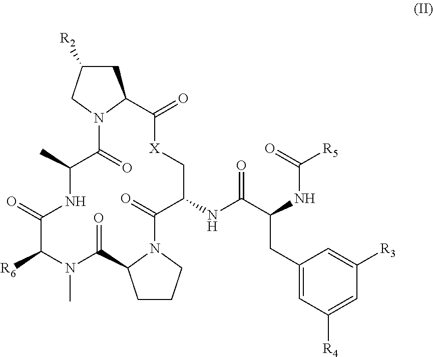

wherein R1, R2, R3, and R4, are each independently H, alkyl, aryl, halogen, and R5 is hydrogen, alkyl, alkenyl, or aralkyl where H may be hydrogen, deuterium, or tritium and wherein R6 can be methyl, ester, or CH₂O(CO)—R7 and R7 can be aryl, azidobenzene, CH₂NH₂

In particular embodiments, R1 is alkyl. In other embodiments, R1 is CH₃. In certain embodiments, R5 is alkyl. In particular embodiments, R5 is an alkyl having one to six carbons. In other embodiments, R3 is fluorine. In other embodiments, R2 and R4 are hydrogen.

In particular aspects disclosed herein, one or more ADEP compounds are used in compositions to eradicate bacterial cell populations. In certain embodiments, ADEP4 and 10c are used to eradicate bacterial cells.

In particular aspects disclosed herein, one or more ADEP compounds are used in compositions to eradicate bacterial cell populations. In certain embodiments, ADEP4 and 10c are used to eradicate bacterial cells.

### 3. ADEP-Antibiotics Compositions

Compositions and methods disclosed herein comprise one or more ADEP compounds in combination with at least one antibiotic. The compositions allow for the use of at least one antibiotic that is active against ADEP-resistant mutants produces a potent bacteria eradicating combination. In particular, multiple antibiotics can be provided in the compositions to form an antibiotic “cocktail.” In such embodiments, each antibiotic is provided in an amount effective to kill a bacterial cell. Exemplary antibiotics include, but are not limited to, from rifampicin, oxacillin, ampicillin, anthracyclin, b-lactam antibiotics, rifamycin group antibiotics (e.g., rifampicin), ciprofloxacin, erythromycin, macrolides (e.g., erythromycin), methicillin, metronidazole, ofloxacin, penicillin, streptomycin, tetracycline, vancomycin, and combinations thereof. In particular embodiments, the antibiotic used in the composition is rifamycin.

In one embodiment, the combination of ADEP compounds and at least one antibiotic is administered in an effective amount to eradicate a bacterial cell population (e.g., treat an infection in a subject or eradicate bacteria from a device or material). In one aspect, ADEP is ADEP 4. In another aspect, ADEP is ADEP 10c. In one aspect, the effective amount of ADEP or antibiotic in combination is 0.5 mgmg to 5,000 mgmg. In another aspect, the effective amount of ADEP or antibiotic in the combination is 0.5 mgmg to 500 mgmg, 0.5 mgmg to 250 mgmg, 0.5 mgmg to 100 mgmg. In another aspect, the effective amount of ADEP or antibiotic in the combination is 0.5 mgmg to 80 mgmg, 0.5 mgmg to 60 mgmg, 0.5 mgmg to 50 mgmg, 0.5 mgmg to 25 mgmg, 0.5 mgmg to 20 mgmg, 0.5 mgmg to 10 mgmg, or 0.5 mgmg to 5 mgmg.

As described above, ADEP compounds are useful in eradicating recalcitrant chronic infections and biofilms. The disclosed compositions and methods allow for treatment or elimination of chronic or relapsing infections by administering an effective amount of ADEP to kill bacteria. In particular, ADEP derivatives such as ADEP 4 and ADEP 10c are particularly useful for eradicating bacteria from devices and treating bacterial infections in accordance with the present disclosure. In one aspect, ADEP, or a derivative thereof, is administered in dosages of about 0.5 mg to about 5,000 mg. In another aspect, the effective amount of ADEP is 0.5 mg to 500 mg, 0.5 mg to 250 mg, 0.5 mg to 100 mg. In another aspect, the effective amount of ADEP or antibiotic in the combination is 0.5 mg to 80 mg, 0.5 mg to 60 mg, 0.5 mg to 50 mg, 0.5 mg to 25 mg, 0.5 mg to 20 mg, 0.5 mg to 10 mg, or 0.5 mg to 5 mg. ADEP derivative, ADEP 4, is administered in similar dosages described herein. In another aspect, the effective amount of antibiotic in the combination is the dose recommended by the manufacturer.

### 4. Methods of Sterilization and Treatment

The methods disclosed herein comprise administering compositions to a subject such as a human to treat infections caused by bacterial infections. In certain embodiments, the methods comprise treating Gram-positive bacterial populations that form biofilms, such as endocarditis, deep-seated infections, catheter-induced infections, and infective osteomyelitis. In other embodiments, the methods comprise treating gram-negative bacteria infections. In particular embodiments, the methods comprise treating Neisseria gonorrhoeae infections.

In certain embodiments, the compositions used in the methods further comprise polymyxin B nonapeptide (“PMBN”). For instance, the compositions include PMBN when treating gram-negative infections. In particular embodiments, the compositions can further include MDR inhibitors.

The presently disclosed methods are effective at killing all types of bacterial cells in a biofilm, i.e., exponentially growing cells, stationary cells, and persister cells. Furthermore, the methods are effective at killing cells that are not present in a biofilm, but growing in a dispersed culture. The methods disclosed herein can specifically treat gram-positive bacteria such as S. aureus.

The effective amount can also be the amount of ADEP in combination with one or more antibiotics that leads to successful treatment of a bacterial infection. In one aspect, the effective amount of ADEP in accordance with the present disclosure can be a dosage of about 0.5 mg to about 5,000 mg per day for a subject. In another aspect, the effective amount of ADEP can be about 250-1000 mg of ADEP. Effective amounts of antibiotics are known to physicians and pharmacists and such information can be obtained from the manufacturer of such antibiotics, or from the Physician's Desk Reference, Medical Economics Co. (published yearly). The compositions can also include about 1.0 ng to about 20 mg of one or more antibiotics. The compositions can also include about 1.0 μg to about 100 μg of one or more antibiotics. In another aspect, the compositions include the dose recommended by the antibiotic manufacturer.

The compositions disclosed herein can be prepared for oral administration. For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use.

The compositions disclosed herein can also be prepared for parenteral administration by injection, e.g., by bolus injection or continuous infusion. Compositions for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Furthermore, disclosed herein are methods of eradicating bacteria from a device. The methods comprise contacting the device with a combination of the ADEP compounds in combination with at least one antibiotic. The combination is effective to kill bacteria present on the device. In particular embodiments, the device is submerged in a solution comprising the composition. In other embodiments, the composition is applied to a surface of the device using a cloth material impregnated with the composition. In other embodiments, the composition is applied to a surface of the device by spray application.

One of ordinary skill in the art will understand that the composition should contact the device for a period of time to allow for the sterilization of the device. Any period of time can be used. For example, sterilization can be achieved in two or fewer days. In certain aspects, sterilization can be achieved in 24 hours or less.

As disclosed above, the compositions can comprise an effective amount of ADEP compounds. For example, ADEP compounds can be provided in concentrations of 0.5 mg to 5,000 mg. In another aspect, the effective amount of ADEP is 0.5 mg to 500 mg, 0.5 mg to 250 mg, 0.5 mg to 100 mg. In another aspect, the effective amount of ADEP is 0.5 mg to 80 mg, 0.5 mg to 60 mg, 0.5 mg to 50 mg, 0.5 mg to 25 mg, 0.5 mg to 20 mg, 0.5 mg to 10 mg, or 0.5 mg to 5 mg. Furthermore, and as disclosed above, the effective amount of one or more antibiotics is 0.5 mg to 5,000 mg. The effective amount of the one or more antibiotics is 0.5 mg to 500 mg, 0.5 mg to 250 mg, 0.5 mg to 100 mg. In another aspect, the effective amount of the one or more antibiotics is 0.5 mg to 80 mg, 0.5 mg to 60 mg, 0.5 mg to 50 mg, 0.5 mg to 25 mg, 0.5 mg to 20 mg, 0.5 mg to 10 mg, or 0.5 mg to 5 mg. In particular embodiments, a plurality of antibiotics is provided in the composition. In such embodiments, each antibiotic is provided in an effective amount. In one aspect, the effective amount of antibiotic in the combination is the dose recommended by the manufacturer.

Additionally, the disclosed compositions are useful in methods of eradicating bacteria from surgical devices. As used herein, the term “surgical device” means a tool designed for performing or carrying out certain actions during surgery on a subject. Surgical devices include scalpels, forceps, hemostats, clamps, retractors, distractors, lancets, drills, rasps, trocars, ligasures, dilators, suction devices, needles, irrigation devices, and implantable devices. Examples of implantable devices include stents, catheters, screws, plates, and other surgical devices designed to be left in the body.

Furthermore the sterilization of devices such as prostheses can be carried out according to the disclosure. Non-limiting examples of devices that can be eradicated according to the present disclosure include a prosthesis (e.g., limb, hip, digit, knee, foot, nasal, auricular, and ocular prosthesis), catheter (e.g., central line, peripherally inserted central catheter (PICC) line, urinary, vascular, peritoneal dialysis, and central venous catheters), catheter connector (e.g., Leur-Lok and needleless connectors), clamp, skin hook, shunt, capillary tube, endotracheal tube, associated ventilator tubing, organ component (e.g., intrauterine device, defibrillator, corneal, and breast), artificial organ or a component thereof (e.g., heart valve, ventricular assist devices, total artificial hearts, cochlear implant, visual prosthetic, and components thereof), dental implant, biosensor (e.g., glucose and insulin monitor, blood oxygen sensor, hemoglobin sensor, biological microelectromechanical devices (bioMEMs), sepsis diagnostic sensor, and other protein and enzyme sensors), bioelectrode, endoscope (hysteroscope, cystoscope, amnioscope, laparoscope, gastroscope, mediastinoscope, bronchoscope, esophagoscope, rhinoscope, arthroscope, proctoscope, colonoscope, nephroscope, angioscope, thoracoscope, esophagoscope, laryngoscope, and encephaloscope), and combinations thereof.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### EXAMPLES

### Example 1

Methods of making ADEP are detailed in U.S. Pat. No. 6,858,585. Moreover, derivatives ADEP 4 and ADEP 10c can be obtained from Wuxi AppTec in St. Paul, Minn.

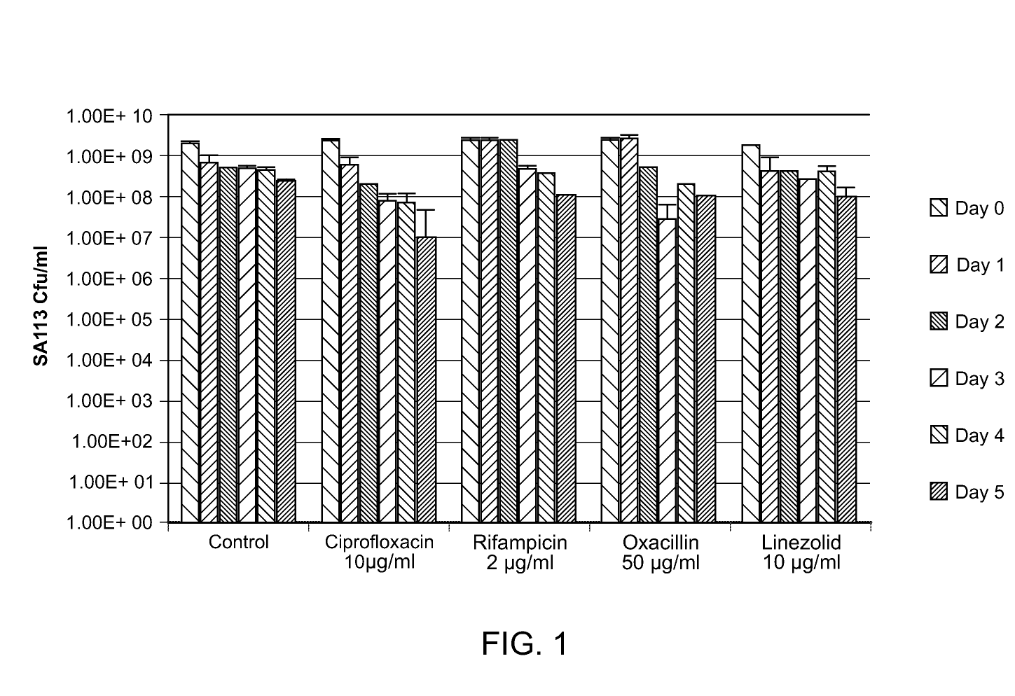

The activity of derivatives ADEP 10c and ADEP 4 were compared to other antimicrobials. ADEP 10c was found to have an S. aureus MIC of 5 μg/ml. ADEP 4 was found to have an MIC of 0.75 μg/ml against S. aureus. Referring to FIG. 1, antibiotic action against stationary state S. aureus. SA113, an MSSA commonly used as a S. aureus model strain, was evaluated. S. aureus SA113 was grown in Mueller-Hinton broth for 24 hours. Antibiotics were added at day 0. Time-points were taken every 24 hours. 100 μl of culture was removed, centrifuged for one minute, and the cells were resuspended in PBS. Serial dilutions from neat to 10⁻⁶ were spotted on MHA plates and incubated overnight at 37° C. The results shown in FIG. 1 are the averages of three independent experiments.

As FIG. 1 shows, bactericidal antibiotics ciprofloxacin and rifampicin had little effect on a stationary population of S. aureus cells after a 5-day incubation period. Daptomycin has previously been shown to have some activity against stationary S. aureus at high concentrations (24 μg/ml) although sterilization has not been reported (Murillo, O., C. Garrigos, M. E. Pachon, G. Euba, R. Verdaguer, C. Cabellos, J. Cabo, F. Gudiol & J. Ariza, (2009) Efficacy of high doses of daptomycin versus alternative therapies against experimental foreign-body infection by methicillin-resistant Staphylococcus aureus. Antimicrob Agents Chemother 53: 4252-4257). Moreover, oxacillin, which only kills growing cells, and linezolid had little effect against S. aureus.

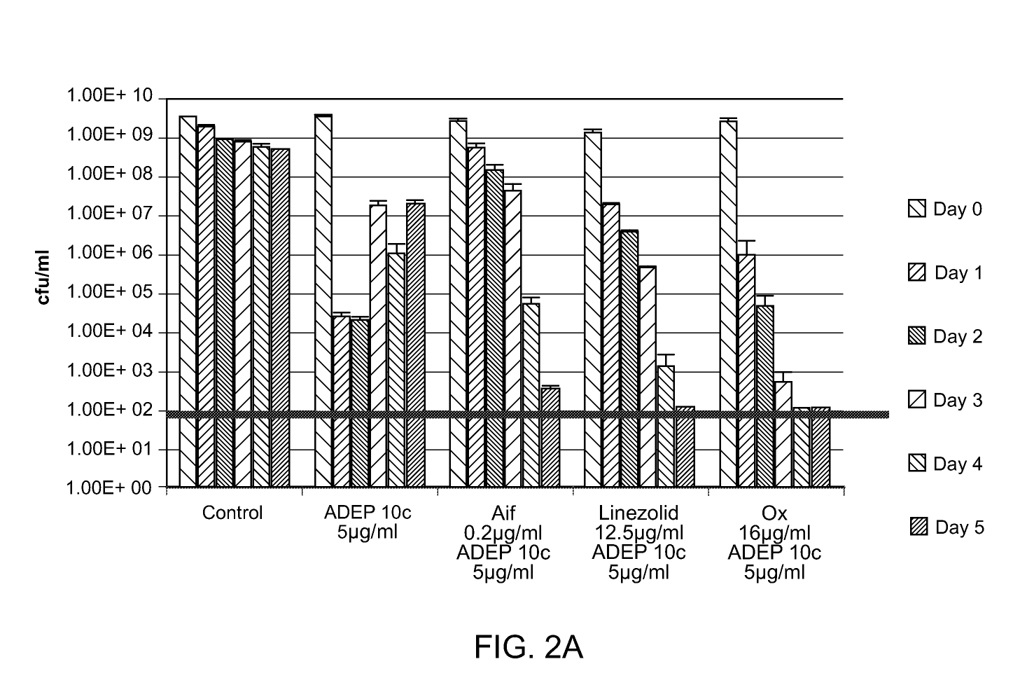

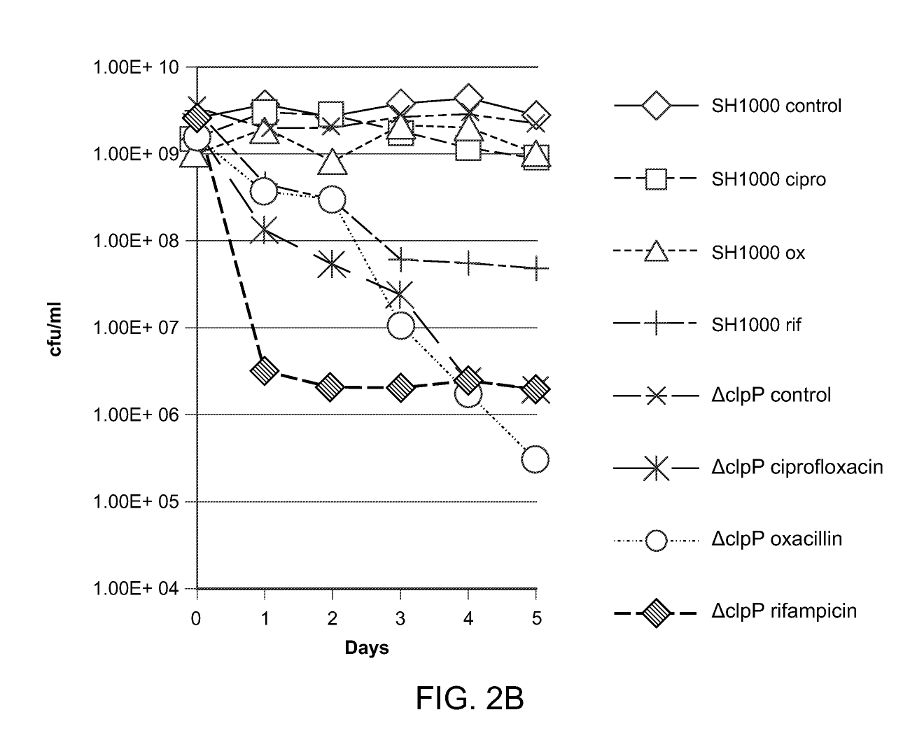

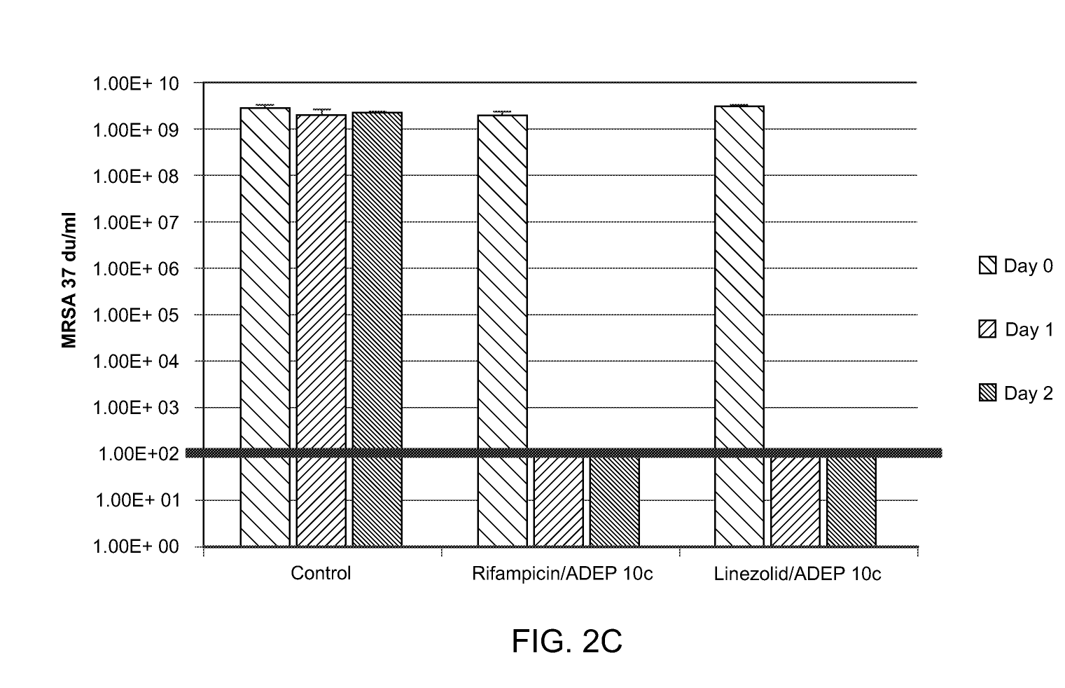

In contrast, the addition of ADEP 10c at 1×MIC produced significant killing after 1 day of incubation, and decreased cell numbers of the stationary population from 10⁹ to about 10⁴ per ml (FIG. 2). After day 1, the culture partially rebounded, likely due to the appearance and growth of clpP mutants (see FIG. 2A). Further, combinations of ADEP 10c with rifampicin, oxacillin and linezolid produced complete sterilization by day 5 (FIG. 2A). Interestingly, the combination of ADEP 10c with vancomycin at 10×MIC prevented re-growth of resistant mutants but did not result in sterilization. This suggests a synergistic effect between ADEP and other antibiotics (not shown).

Surprisingly, complete sterilization was achieved in all of the ADEP-antibiotic combinations, including ADEP in combination with bacteriostatic linezolid. The susceptibility of a clpP mutant to these antibiotics was investigated. It was found that each of the antibiotics tested showed considerable killing of stationary clpP cells (FIG. 2B). The MIC of the clpP strain exposed to the antibiotics was the same as that for the wild type. Apparently, clpP mutants have decreased fitness which manifests as elevated susceptibility to killing by antibiotics. Thus, once a resistant clpP mutant is formed in vivo during treatment, it will be killed by the second antibiotic in the combination.

The ability of ADEP 10c to kill an S. aureus MRSA strain was also evaluated. Surprisingly, a combination of ADEP 10c with rifampicin or linezolid produced complete sterilization after 24 hours of incubation (FIG. 2C).

Resistance to methicillin and oxacillin is the hallmark of MRSA. The inability to use these effective antibiotics narrows MRSA treatment options. As expected, oxacillin had no effect on killing stationary MRSA cells. However, in the presence of ADEP 10c, oxacillin at a clinically achievable concentration (16 μg/ml) prevented the rise of clpP mutants. The combination was also effective at killing stationary cultures even though complete sterilization was not achieved (not shown). Oxacillin killed clpP mutants of MRSA and restored oxacillin susceptibility of this MRSA strain, which has an oxacillin MIC >75 μg/ml.

### Example 2

ADEP 4 has an S. aureus IC 50 of 0.05 μg/ml (Brotz-Oesterhelt, H., D. Beyer, H. P. Kroll, R. Endermann, C. Ladel, W. Schroeder, B. Hinzen, S. Raddatz, H. Paulsen, K. Henninger, J. E. Bandow, H. G. Sahl & H. Labischinski, (2005) Dysregulation of bacterial proteolytic machinery by a new class of antibiotics. Nat Med 11: 1082-1087). It was determined that MIC of ADEP 10c is 5 μg/ml, and MIC of ADEP 4 is 0.75 μg/ml when tested with a variety of MSSA and MRSA isolates. ADEP 4 at 1.5×MIC showed no killing activity against stationary S. aureus after 24 hours. However, when combined with rifampicin, ADEP 4 resulted in complete sterilization in 5 days (not shown).

### Example 3

Evaluation of ADEP 10c and ADEP 4 showed that ADEP 10c had a notably higher MIC than ADEP 4 compound. An activity-based SAR of ADEP compounds has been previously reported (Brotz-Oesterhelt, H., D. Beyer, H. P. Kroll, R. Endermann, C. Ladel, W. Schroeder, B. Hinzen, S. Raddatz, H. Paulsen, K. Henninger, J. E. Bandow, H. G. Sahl & H. Labischinski, (2005) Dysregulation of bacterial proteolytic machinery by a new class of antibiotics. Nat Med 11: 1082-1087). A number of analogs were examined herein to obtain an SAR that informs not only potency but also killing ability.

Analogs that show the superior eradicating activity while retaining good potency, MIC≦1 μg/ml, are good candidates for development. Approximately 40 derivatives of the natural products enopeptin A or B have been described and assessed for their antibacterial activity (Brotz-Oesterhelt, H., D. Beyer, H. P. Kroll, R. Endermann, C. Ladel, W. Schroeder, B. Hinzen, S. Raddatz, H. Paulsen, K. Henninger, J. E. Bandow, H. G. Sahl & H. Labischinski, (2005) Dysregulation of bacterial proteolytic machinery by a new class of antibiotics. Nat Med 11: 1082-1087).

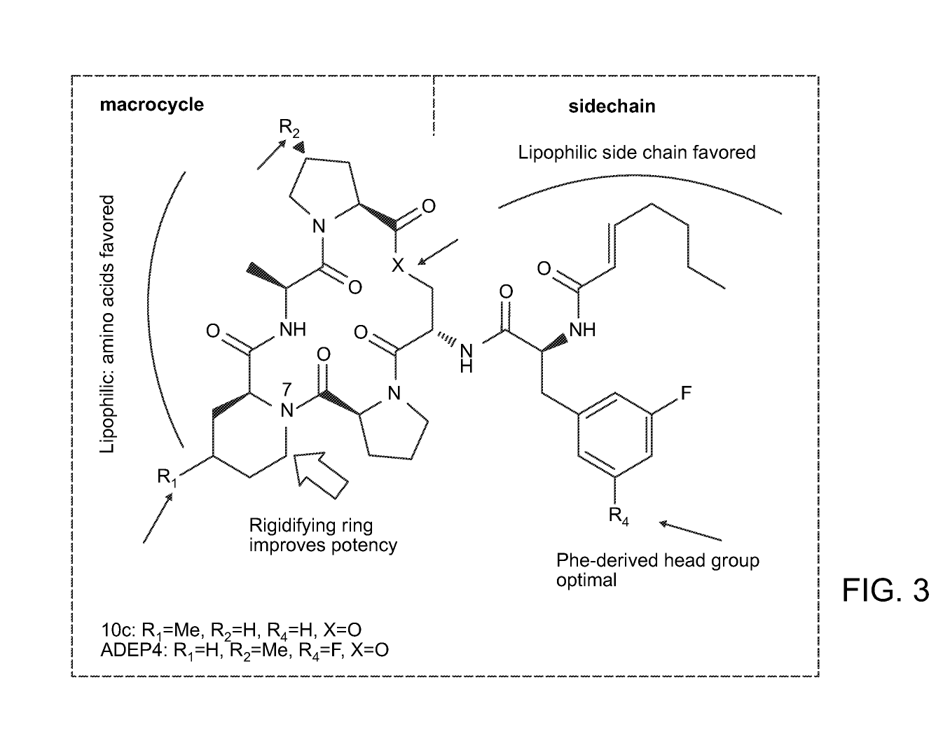

Referring to FIG. 3, published SAR studies have explored both the macrocyclic core and the acyl sidechain. Overall, an optimal core macrocycle consists of five lipophilic (S)-amino acids, where the serine amine is acylated with a phenylalanine derivative and capped with a fatty acyl tail of discreet chain length and lipophilicity. Acyl side chains consisting of phenylalanine derivatives were the most active, where inversion of the chiral center and replacement of the phenyl with other heterocycles decreased or abrogated activity. The 3,5-difluoro and 3-F compounds appeared to be most active, and changes to the capping acyl group affected activity. Sidechain lengths of 1-6 carbons were most active and β unsaturated derivatives with a trans double bond favored.

In studies of the macrocycle of the original natural products, modification of the Northern proline decreased activity and N7 alkylation was essential whereby the methyl group maintained activity. Rigidification at the N7 position via heterocycle substructures resulted in increased activity compared to the N7 methyl derivative, where the piperidine substructure was the most potent compound derived. This was the most potent compound, which showed superior activity in a murine model of MRSA infection compared to linezolid.

Although the MIC of ADEP 4 is lower than that of ADEP 10c, it was determined that ADEP 10c is more efficacious at killing stationary cells. Without wishing to be bound to a particular theory, it is speculate that, based on the qualitative differences in positioning of lipophilic functionality (i.e. methylation, fluorination), this unexpected result may be due to the difference in cellular permeability of growing cells versus stationary cells.

### Example 4

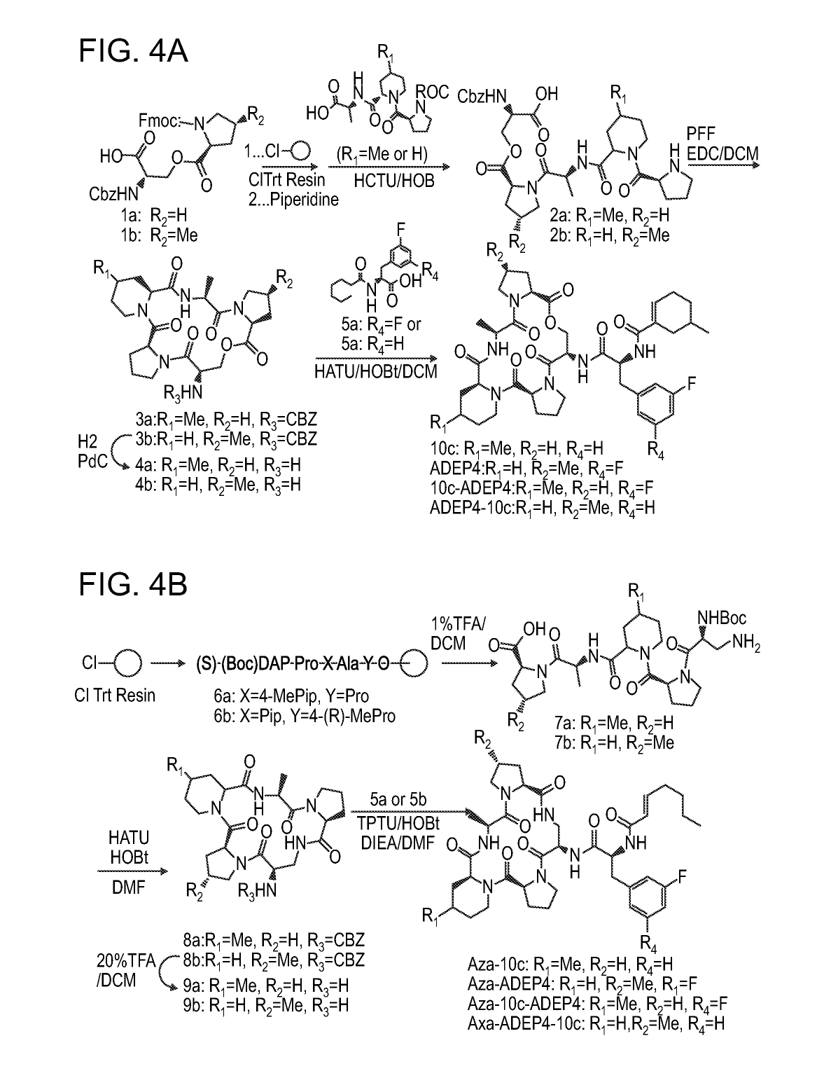

Chemical synthesis of eradicating ADEP derivatives was investigated. The chemical structure of ADEP can be dissected into two regions, the macrocycle, and the side chain (see FIG. 3). As FIG. 3 shows, ADEP 4 and ADEP 10c differ by the R-group substituents noted with a line arrow. The differences observed in the MIC and the killing activity between these compounds led to the design of a set of crossover analogs that match the ADEP 4 head group with the ADEP 10c side chain, and the ADEP 10c head group with the ADEP 4 side chain (FIG. 4A).

The synthesis of ADEP 4 and ADEP 10c was performed using the chemical methodology shown in FIG. 4. Construction of a linear peptide (2) using resin-bound methodology was followed by macrocyclization via activated ester formation. Hydrogenolytic deprotection of the primary amine affords 4, which was coupled with the desired sidechain carboxylic acid (5).

This methodology was adapted to make aza-analogs (FIG. 4B). Construction of the linear peptide was achieved on-resin. The compound was cleavage with 1% trifluoroacetic acid and macrocyclization using standard conditions provides macrocycle 8, which can be de-protected and acylated with acids 5. This resulted in the formation of new analogs Aza-ADEP 4 and Aza-ADEP 10c.

This method can be used to synthesize other macrocyclic peptide analogs by modifying the desired amino acids in the solid-phase synthesis process. A wide variety of sidechain analogs can be prepared from the deprotected macrocycles 4 and 9 by reacting with various electrophilic reagents (isocyanates, carboxylic acids, sulfonyl chlorides, etc.) to allow further rapid exploration in this region.

### Example 5

Eradicating combinations of lead ADEP compounds and antibiotics were evaluated for their ability to eradicate stationary and biofilm populations of pathogens.

Rifampicin is the most effective of available antibiotics for treating biofilm infections of indwelling devices and osteomyelitis, and is usually administered in combination with another antibiotics due to the high probability of resistance development. The combination of ADEP and rifampin successfully eradicated stationary S. aureus.

Experiments with exponentially growing and stationary cultures are performed using compounds at their clinically achievable concentrations, which are 7.2 μg/ml for rifampicin, 14.5 μg/ml for linezolid and 16 μg/ml for oxacillin (Chik, Z., R. C. Basu, R. Pendek, T. C. Lee & Z. Mohamed, (2010) A bioequivalence comparison of two compositions of rifampicin (300-vs 150-mg capsules): An open-label, randomized, two-treatment, two-way crossover study in healthy volunteers. Clinical therapeutics 32: 1822-1831; Burkhardt, O., K. Borner, N. von der Hoh, P. Koppe, M. W. Pletz, C. E. Nord & H. Lode, (2002) Single- and multiple-dose pharmacokinetics of linezolid and co-amoxiclav in healthy human volunteers. J Antimicrob Chemother 50: 707-712; Glew, R. H. & R. C. Moellering, Jr., (1979) Effect of protein binding on the activity of penicillins in combination with gentamicin against enterococci. Antimicrob Agents Chemother 15: 87-92).

ADEP at 1×MIC was found to be highly effective in preliminary studies. According to PK data for ADEP 4, its achievable concentration is 7.5 μg/ml (10×MIC).

As previously noted, stationary cultures are more tolerant to antibiotics than slowly growing biofilms (Spoering, A. L. & K. Lewis, (2001) Biofilms and planktonic cells of Pseudomonas aeruginosa have similar resistance to killing by antimicrobials. J Bacteriol 183: 6746-6751). Therefore, it is expected that combinations of compounds that effectively eradicate stationary populations will be even more effective against biofilms.

To evaluate biofilms, two models are used. One model used is a Calgary™ device with prongs that are placed in a suspension of bacteria in nutrient medium. Biofilms are allowed to form. The platform is then placed in a 96 well plate with antibiotics. After an incubation period, biofilms are dislodged by mild sonication. The cells are resuspended by vortexing and are then plated for cfu counts.

The second model used to evaluate biofilms more closely resembles an environment in vivo. Briefly, a suspension of bacteria is injected into capillary tubes, which are left stagnant to allow cells to attach to the capillary surface. The capillaries are then rinsed with sterile water to remove loosely bound cells. A peristaltic pump is then used to pump fresh medium through the tubes for 24-48 hours at 37° C. The attached cells proliferate, and a biofilm forms on the interior of the tube. Sterile water is then pumped through the capillaries to remove loosely bound cells. Fresh medium containing ADEP and rifampicin are passed through these chambers for 24 hours. Biofilms are stained using LIVE/DEAD stain and analyze for viability using microscopy. Image analysis software is used to quantify the relative number of live versus dead cells.

### Example 6

PK and MTD studies are performed to determine the dose of a eradicating combination for testing sterilization in vivo. The eradicating combination is tested in a mouse model of a S. aureus biofilm infection. Existing antibiotics do not produce clearance of infection in this model. Sufficient clearance by the combination as compared to a benchmark comparator, vancomycin, constitutes proof-of-principle for this developmental therapeutic.

The default formulation for in vivo studies is saline, as early experience with the series indicates good solubility. However, additional compositions, such as 5% DMSO, 5% ethanol, or beta-cyclodextrin, are also tested on compounds showing poor solubility.

Pharmacokinetics. Firstly, pharmacokinetic (PK) blood levels are determined following intraperitoneal dosing. Mice are dosed IP and blood is withdrawn from the tail vein at 10, 20 and 30 minutes post-dose. At 45 minutes post-dose, the mice are euthanized and bled out by cardiac puncture. Each compound is dosed at 5 mg/kg and 50 mg/kg to determine whether PK is linear (10 mg of compound). Compounds are quantified using LCMS. Compounds showing a high free AUC or a long half-life are selected.

To determine the penetration of drug into the tissue cage, samples are removed from the tissue cages of the 50 mg/kg rifampicin and 50 mg/kg lead compound groups just prior to the second and last dose of the study to determine trough concentrations, and 4 hours later to determine peak concentrations.

Efficacy. The lead compound progresses to the in vivo biofilm model (Kristian, S. A., X. Lauth, V. Nizet, F. Goetz, B. Neumeister, A. Peschel & R. Landmann, (2003) Alanylation of teichoic acids protects Staphylococcus aureus against Toll-like receptor 2-dependent host defense in a mouse tissue cage infection model. J Infect Dis 188: 414-423). Others using this model in rats have been unable to clear a staphylococcal biofilm with three broad-spectrum antibiotics dosed either alone or in combination (Lucet, J. C., M. Herrmann, P. Rohner, R. Auckenthaler, F. A. Waldvogel & D. P. Lew, (1990) Treatment of experimental foreign body infection caused by methicillin-resistant Staphylococcus aureus. Antimicrob Agents Chemother 34: 2312-2317).

However, based on the in vitro data disclosed herein, it is anticipated that a combination of rifampicin and a lead ADEP compound will clear the biofilm in this model. Briefly, a sterile tissue cage containing sintered glass beads is implanted subcutaneously in the back of the anaesthetized mouse. Two weeks after surgery, the cage is verified to be sterile, the mice are then immunocompromized, 200 μL of a S. aureus culture is introduced into the cage and the animals are left for 14 days to allow the infection to stabilize. Mice are then dosed for 7 days and euthanized to allow removal of the tissue cage. Bacterial counts are then performed on the cage fluid and on the glass beads following washing and sonication of the beads.

The combinations of rifampicin (25 mg/kg) and vancomycin (50 mg/kg) with two test combinations, and rifampicin (25 mg/kg) in combination with the lead ADEP compound (50 & 25 mg/kg, respectively) are assessed. An infected untreated control group is also assessed. If the lead ADEP compound in combination with rifampicin proves the superior combination as evidenced by a lower bacterial count on glass beads, the ratios of the two agents will be optimized for sterilization.

1. A method of treating a bacterial infection, the method comprising:
(a) administering to a subject an effective amount of a compound having the structure: 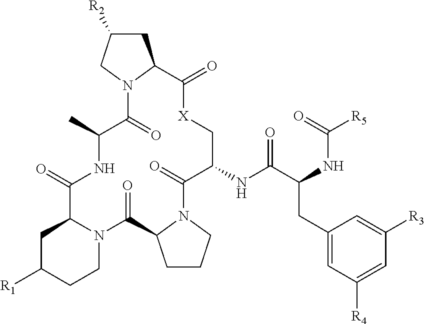
wherein R1, R2, R3, and R4 are each independently H, alkyl, aryl, or halogen,
R5 is hydrogen, alkyl, alkenyl, or aralkyl where H may be hydrogen, deuterium, or tritium,
wherein X is oxygen or NH, or a pharmaceutically acceptable salt thereof; and
(b) administering an effective amount of one or more antibiotics in combination with the compound, wherein the combination of the compound and one or more antibiotics kills bacterial cells.

2. The method of claim 1, wherein the subject is treated for at least 2 days with the combination.

3. The method of claim 1, wherein the one or more antibiotics are selected from rifampicin, oxacillin, amphotericin, ampicillin, b-lactam antibiotics, rifamycin group antibiotics, ciprofloxacin, erythromycin, macrolides, methicillin, metronidazole, ofloxacin, penicillin, streptomycin, tetracycline, vancomycin, and combinations thereof.

4. The method of claim 1, wherein the bacterial cells are resistant to an acyldepsipeptide.

5. The method of claim 1, wherein the bacterial cells are persister cells.

6. The method of claim 1, wherein the bacterial cells are persister cells, cells in stationary growth phase, or rapidly growing cells.

7. The method of claim 1, wherein the bacterial cells are gram positive.

8. The method of claim 1, wherein the bacterial cells are selected from MRSA S. aureus, VRE E. faecalis, S. pneumoniae, S. epidermidis, and combinations thereof.

9. The method of claim 1, wherein the bacterial cells are gram-negative.

10. The method of claim 9, wherein the composition further comprises polymyxin B nonapeptide.

11. The method of claim 9, wherein the composition further comprises MDR inhibitor.

12. A method of eradicating bacteria from a device, the method comprising:
(a) contacting the device with a compound having the structure: 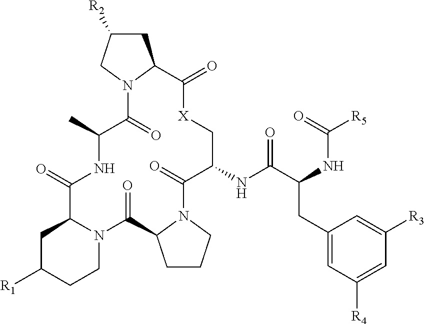
wherein R1, R2, R3, and R4 are each independently H, alkyl, aryl, or halogen,
R5 is hydrogen, alkyl, alkenyl, or aralkyl where H may be hydrogen, deuterium, or tritium,
wherein X is oxygen or NH, or a pharmaceutically acceptable salt thereof, and
(b) contacting the device with at least one antibiotic,
wherein the combination of the compound and at least one antibiotic is effective to kill the bacteria on the device.

13. The method of claim 12, wherein the at least one antibiotic is selected from rifampicin, oxacillin, ampicillin, b-lactam antibiotics, rifamycin group antibiotics, ciprofloxacin, erythromycin, macrolides, methicillin, metronidazole, ofloxacin, penicillin, streptomycin, tetracycline, vancomycin, and combinations thereof.

14. The method of claim 12, wherein the device is an implantable device.

15. The method of claim 12, wherein the combination comprises an effective amount of the compound and an effective amount of at least one antibiotic to eradicate bacteria from the device.

16. The method of claim 12, wherein the bacterial cells are gram-negative.

17. The method of claim 16, wherein the composition further comprises polymyxin B nonapeptide.

18. The method of claim 16, wherein the composition further comprises MDR inhibitor.

19. A formulation for killing persister bacterial cells comprising:
a combination of an effective amount of a compound having a structure: 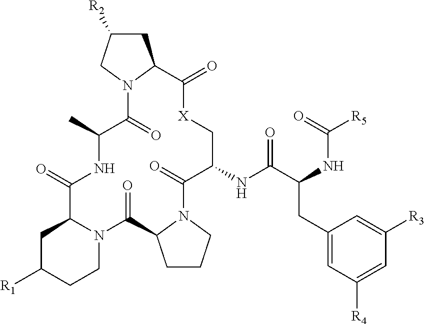
wherein R1, R2, R3, and R4 are each independently H, alkyl, aryl, or halogen,
R5 is hydrogen, alkyl, alkenyl, or aralkyl where H may be hydrogen, deuterium, or tritium,
wherein X is oxygen or NH, or a pharmaceutically acceptable salt thereof and an effective amount of at least one antibiotic.

20. The formulation of claim 19, wherein the effective amount of the compound is selected from the range of 0.5 mg/ml to 250 mg/ml.

21. The formulation of claim 19, wherein the effective amount of the at least one antibiotic is selected from the range of 0.5 mg/ml to 250 mg/ml.

22. The formulation of claim 19, wherein the at least one antibiotic is selected from rifampicin, oxacillin, ampicillin, b-lactam antibiotics, rifamycin group antibiotics, ciprofloxacin, erythromycin, macrolides, methicillin, metronidazole, ofloxacin, penicillin, Streptomycin, tetracycline, vancomycin, and combinations thereof.

23. The formulation of claim 20, wherein the at least one antibiotic is rifamycin.

24. The formulation of claim 23, wherein the compound is ADEP4.

25. The formulation of claim 24, wherein the effective amount of the at least one antibiotic is 0.5 mg/ml to 250 mg/ml.

26. The formulation of claim 23, wherein the compound is L-proline, 3-fluoro-N-[(2E)-1-oxo-2-hepten-1-yl]-L-phenylalanyl-L-seryl-L-prolyl-(2S)-4-methyl-2-piperidinecarbonyl-L-alanyl-, (6→2)-lactone.

27. The formulation of claim 19 further comprising polymyxin B nonapeptide.

28. The method of claim 19 further comprising MDR inhibitor.

29. A method of eradicating a persister bacterial population, the method comprising:
(a) administering to a subject an effective amount of a compound having the structure: 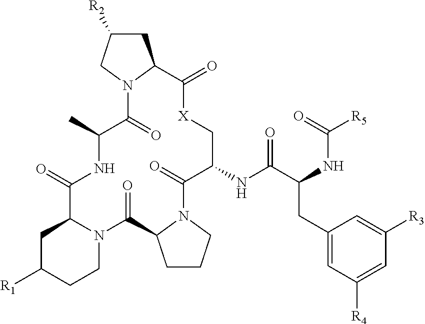
wherein R1, R2, R3, and R4 are each independently H, alkyl, aryl, or halogen,
R5 is hydrogen, alkyl, alkenyl, or aralkyl where H may be hydrogen, deuterium, or tritium, wherein X is oxygen or NH, or a pharmaceutically acceptable salt thereof; and wherein administration of the compound eradicates the persister bacterial cells.

30. The method of claim 29, wherein the subject is treated for at least 2 days with the compound.

31. The method of claim 29, wherein the effective amount of the compound is selected from the range of 0.5 mg/ml to 250 mg/ml.

32. The method of claim 29, wherein the bacterial cells are gram positive.

33. The method of claim 29, wherein the bacterial cells are selected from MRSA S. aureus, VRE E. faecalis, S. pneumoniae, S. epidermidis, and combinations thereof.

34. The method of claim 29, wherein the bacterial cells are gram-negative.

35. The method of claim 34, wherein the composition further comprises polymyxin B nonapeptide.

36. The method of claim 34, wherein the composition further comprises MDR inhibitor.
